(19) United States
(12) Patent Application Publication
(10) Pub. No.: US 2004/0167560 A1
(43) Pub. Date: Aug. 26, 2004

(54) Single-use tattooing and dermopigmentation device

(76) Inventor: Sylvie Merkel, Fleury-La-Riviere (FR)

Correspondence Address: JACOBSON HOLMAN PLLC, 400 SEVENTH STREET N.W., SUITE 600, WASHINGTON, DC, 20004, US

(21) Appl. No.: 10/478,204

(22) PCT Filed: May 28, 2002
(86) PCT No.: PCT/FR02/01796

(30) Foreign Application Priority Data
May 28, 2001 (FR) 01/07026

(51) Int. Cl. A61B 17/34

(52) U.S. Cl. 606/186

(57) ABSTRACT

The invention relates to a single-use tattooing and dermopigmentation device comprising a cylindrical tube (2) with a sleeve to which it is joined (4) and a rod supporting a needle (7) in the said tube. The rod supporting the needle (7) comprises a beaming means (10) on the inner wall (11) of the cylindrical tube (2).

The present invention relates to a single-use tattooing and dermo-pigmentation appliance. Traditionally, such an appliance comprises, on the one hand, a cylindrical tube provided with a sleeve and, on the other hand, a needle-carrying rod arranged in this tube: an example of this appliance is described notably in the document EP-374 355-A.

The tattooing operation should offer complete health safety: this is why it must be possible to sterilise it. For this reason the tattooing appliances currently on the market are produced in a material that can withstand sterilisation. This material is usually stainless steel. Nevertheless, it is not possible by cleaning and decontaminating such an appliance to ensure total reliability in relation to the destruction of pathogenic agents such as, in particular, the tubercle bacillus and the HIV virus.

In addition, the material used has another drawback in that centring the needles for precision work, without oscillation, is not completely guaranteed.

One of the objects of the present invention is therefore to provide a single-use tattooing and dermo-pigmentation appliance that can guarantee its complete sterility. Another object of the invention is to provide such an appliance that makes it possible to ensure precision work without oscillation of the needle-bearing rod in the cylindrical tube.

These objects, together with others that will appear hereafter, are achieved by a single-use tattooing and dermo-pigmentation appliance comprising, on the one hand, a cylindrical tube provided with a sleeve with which it is integral and, on the other hand, a needle-carrying rod arranged in this tube, which appliance is characterised, according to the present invention, by the fact that the needle-carrying rod comprises support means on the internal wall of the cylindrical tube.

Advantageously, these support means comprise at least three fins extending outwards round the needle-carrying rod and arranged at regular intervals thereon.

Preferably, the width of the fins is such that their envelope is the internal wall of the cylindrical tube.

Advantageously, the fins extend over a height corresponding substantially to that of the sleeve.

The description that follows and that is in no way limiting, should be read in relation to the accompanying drawings, in which:

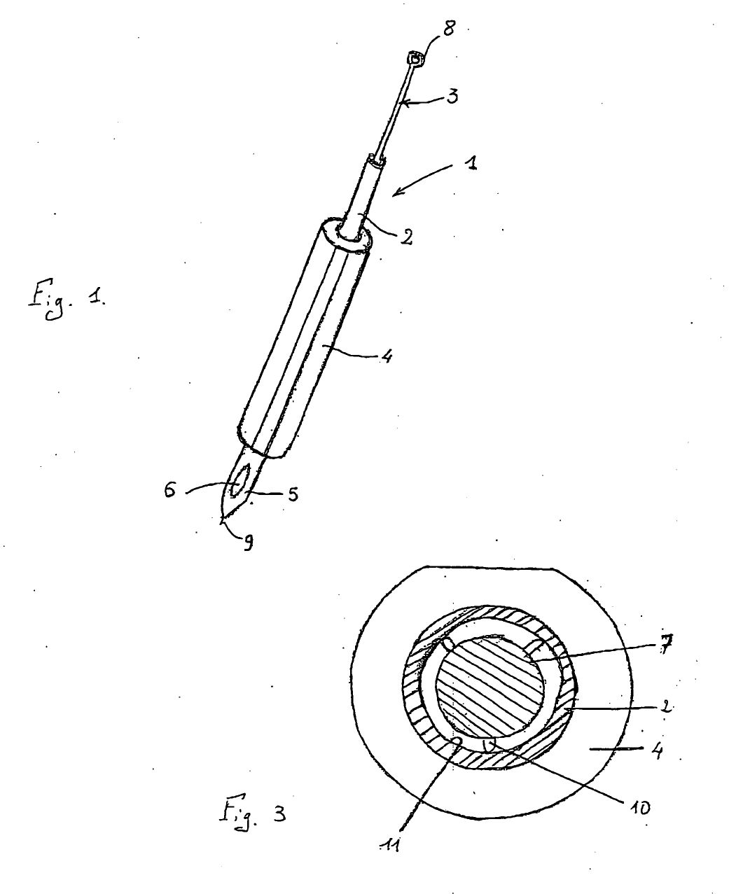

FIG. 1 is a view in perspective of an appliance in accordance with the present invention;

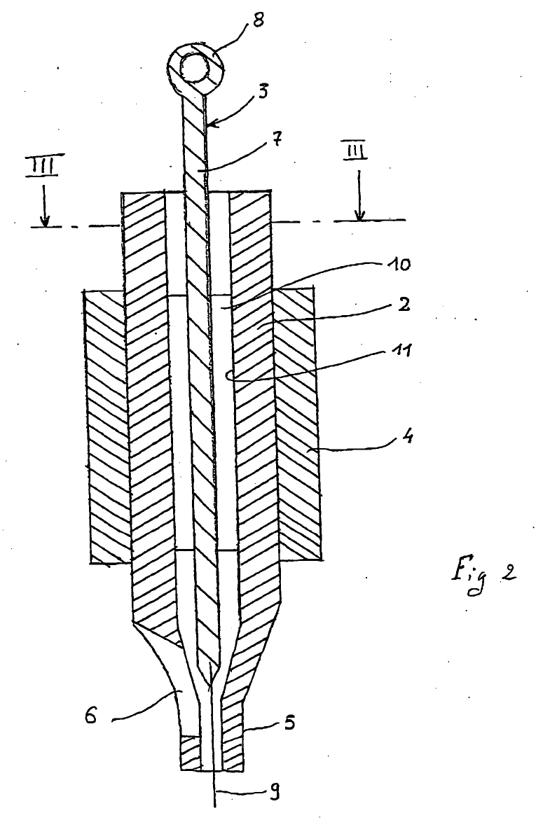

FIG. 2 is a plan view of the cylindrical tube and the needle-carrying rod, which constitute the appliance illustrated in FIG. 1; and,

FIG. 3 is view in section of the needle-carrying rod along the line III-III of FIG. 2.

As illustrated in FIGS. 1 and 2, a single-use tattooing and dermo-pigmentation appliance, designated in its entirety by the reference 1, comprises, on the one hand, a cylindrical tube 2 and, on the other hand, a needle-carrying rod 3 arranged in this tube 2.

The tube 2 comprises a sleeve 4 of standard or variable external diameter which has a flat part for gripping, as is known. Moreover, this sleeve 4 is integral with the tube 2 by any known means. It is extended at one of its ends by an end piece 5 of known form depending on the grouping of the needles. This end piece comprises an opening 6 for the capillary action; the opening also allows the ink level to be displayed.

The needle-carrying rod 3 comprises a rod 7 comprising, at one end, known as the upper end, a standard loop 8 or notch and, at its other end, known as the lower end, known means for fixing at least one needle 9. In particular, this lower end is, depending on the grouping of the needles, rounded or flat in order to receive the bundle of needles which is inserted and stuck in this part of the rod 7. These needles 9 are located in the axis of the rod 7 and have been sterilised in manufacture.

In accordance with the present invention, the rod 7 comprises support means 10 on the internal wall 11 of the cylindrical tube 2. These support means comprise at least three fins 10, which are distributed at regular intervals round the rod 7 and have a width such that the internal wall 11 of the cylindrical tube 2 is their envelope.

The fins 10 therefore extend over a height that corresponds, at the most, substantially to that of the cylindrical tube 2: preferably over the height of the sleeve 4.

During tattooing and dermo-pigmentation operations, the appliance in accordance with the present invention is arranged in a known device driving the rod 7 in a rectilinear back and forth movement: with the aid of the fins 10, the rod 7 is maintained in movement along the same axis, which allows the needles 9 to follow an exact line on the skin of the patient.

1. A single-use tattooing and dermo-pigmentation appliance comprising, on the one hand, a cylindrical tube (2) provided with a sleeve (4) with which it is integral and, on the other hand, a needle-carrying rod (7) arranged in said tube (2) and comprising support means (10) on the internal wall (11) of said cylindrical tube (2), characterised in that these support means comprise at least three fins (10) extending outwards round the needle-carrying rod (7) and arranged at regular intervals thereon, having a width such that the internal wall (11) of the cylindrical tube (2) are their envelope and extending over a height corresponding substantially to that of the sleeve (4).
